(19) United States
(12) Patent Application Publication
(10) Pub. No.: US 2011/0064660 A1
(43) Pub. Date: Mar. 17, 2011

(54) DEUTERATED ALKYL PHOSPHOLIPID COMPOUNDS, COMPOSITIONS, AND METHODS OF USE

(76) Inventors: Anatoly Pinchuk, Madison, WI (US); Jamey P. Weichert, Fitchburg, WI (US); Marc Longino, Verona, WI (US); William R. Clarke, Colgate, WI (US)

(21) Appl. No.: 12/879,093

(22) Filed: Sep. 10, 2010

Related U.S. Application Data
(60) Provisional application No. 61/241,751, filed on Sep. 11, 2009.

(51) Int. Cl. A61K 51/04; C07F 9/09; A61K 31/661; A61K 49/00; A61P 35/00

(52) U.S. Cl. 424/1.77; 564/15; 514/77; 424/9.1

(57) ABSTRACT

The invention generally relates to novel deuterated phospholipid compounds, compositions comprising these compounds, and their use in a variety of cancer therapy and diagnostic applications.

### FIELD OF THE INVENTION

The invention generally relates to deuterated tumor-selective alkyl phospholipid compounds, compositions comprising these compounds, and methods of using these compounds for the treatment and/or diagnosis of various solid cancers.

### BACKGROUND

Alkyl phospholipid compounds and their use in cancer treatment and diagnosis are known in the art. See, for example, U.S. Pat. No. 6,417,384 B1 and WO 2007/013894 A2. In particular, compound CLR1404 (18-(p-iodophenyl)octadecyl phosphocholine) is known and is currently undergoing clinical trials for treatment of various solid cancers.

It would be useful to improve alkyl phospholipid compounds by substituting hydrogen with deuterium at various specific positions in the alkyl phospholipid compounds. Accordingly, the need exists to further improve the alkyl phospholipid compounds.

### SUMMARY OF THE INVENTION

The invention generally relates to deuterated alkyl phospholipid compounds, compositions comprising these compounds, and the use of the compounds and/or compositions in treatment and/or diagnosis of various malignancies.

In one embodiment, the present invention relates to the following deuterated alkyl phospholipid compounds of Formulas 1-6:

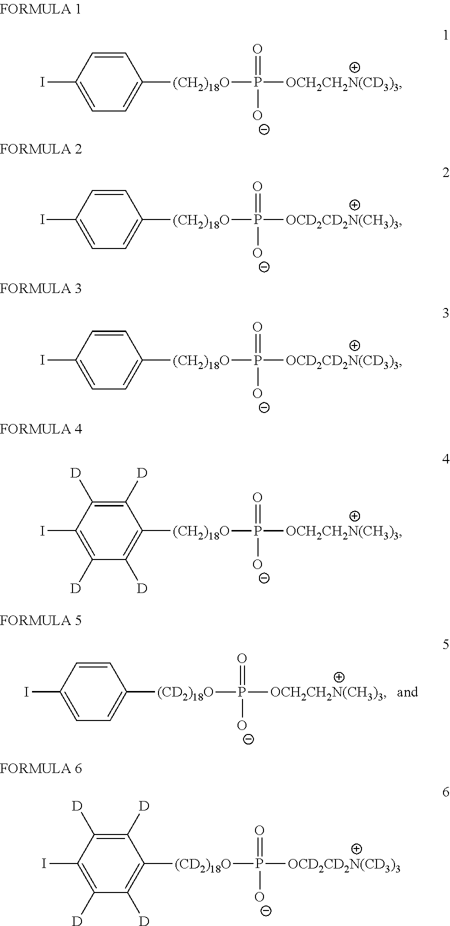

wherein I is a radioactive or stable isotope of iodine.

For a therapy in humans, a preferred radioactive isotope of iodine is ¹³¹I, although other radioactive isotopes, including ¹²³I, ¹²⁴I, and ¹²⁵I can also be used.

In the most preferred embodiment for therapeutical use, the radiolabeled compound is the compound according to Formula 1:

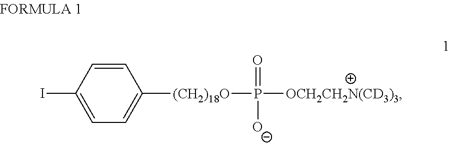

wherein I is ¹³¹I.

For diagnostic use, a preferred radioactive isotope of iodine is ¹²⁴I, although other radioactive isotopes, including ¹²³I and ¹³¹I can be used, too.

In the most preferred embodiment for diagnostic use, the radiolabeled compound is the compound according to Formula 1:

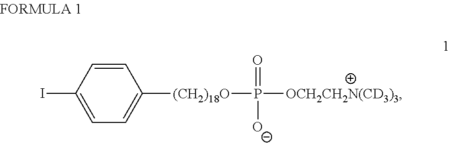

wherein I is ¹²⁴I.

In another embodiment, the invention relates to the deuterated compounds of Formulas 1-6 wherein I is a stable isotope of iodine.

The invention also generally relates to compositions comprising the compounds of the present invention.

The invention also generally relates to various methods of using the compounds of the present invention, including, but not limited to, solid cancer therapy, endoscopic determination of the presence of internal malignancy; visual and/or microscopically added determination of the presence of malignant lesions on the skin; aiding in the selection of biopsy tissues in internal and skin malignancies; determination of the presence of internal and/or skin malignancies during surgeries to aid the complete biopsy and/or surgical resection of said malignancies.

Preferred solid cancers that can be treated with deuterated compounds of the present invention include lung cancer, squamous cell carcinoma, renal cancer, adrenal cancer, melanoma, colon cancer, colorectal cancer, ovarian cancer, prostate cancer, liver cancer, intestinal cancer, hepatocellular carcinoma, retinoblastoma, cervical cancer, glioma, breast cancer, and pancreatic cancer.

### DETAILED DESCRIPTION OF THE INVENTION

In one embodiment, the present invention generally relates to deuterated alkyl phospholipid compounds and various methods of their use for cancer treatment and diagnosis.

In one embodiment, the invention generally refers to the following deuterated alkyl phospholipid compounds of Formulas 1-6:

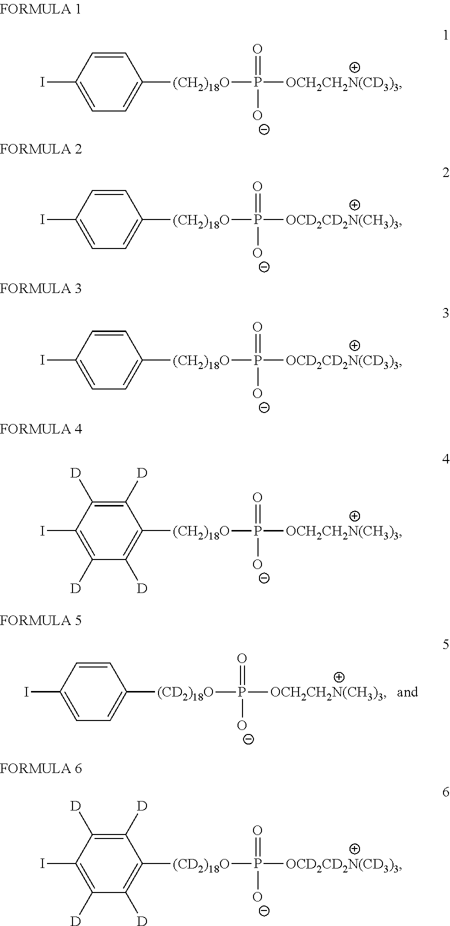

wherein I is a radioactive or stable isotope of iodine.

“ID” refers to deuterium.

For a therapy in humans, a preferred radioactive isotope of iodine is ¹³¹I, although other radioactive isotopes, including ¹²³I, ¹²⁴I, and ¹²⁵I can also be used.

In the most preferred embodiment for therapeutical use, the radiolabeled compound is the compound according to Formula 1:

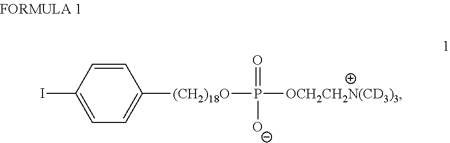

wherein I is ¹³¹I.

For diagnostic use, a preferred radioactive isotope of iodine is ¹²⁴I, although other radioactive isotopes, including ¹²³I and ¹³¹I can be used, too.

In the most preferred embodiment for diagnostic use, the radiolabeled compound is the compound according to Formula 1:

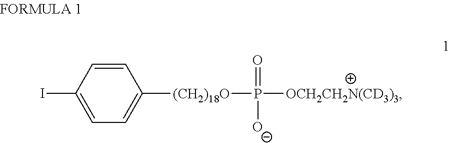

wherein I is ¹²⁴I.

In another embodiment, the invention relates to the deuterated compounds of Formulas 1-6 wherein I is a stable isotope of iodine.

Deuterium can be incorporated to the specific positions in the compounds of Formulas 1-6 synthetically, according to the synthetic procedures known in the art, by using appropriate deuterated intermediates. If a protecting group is used, the synthetic method includes an additional step for the removal of the protecting group. These deuterated intermediates can be prepared by methods known to one of skill in the art. Specific examples of making the deuterated compounds of the present invention are provided in the “Examples” section of the application.

If the specific positions have an exchangeable proton, then the deuterium can be incorporated via proton-deuterium equilibrium exchange. To introduce deuterium, these protons may be replaced with deuteriums selectively or non-selectively through a proton-deuterium exchange method known in the art.

It is to be understood that the present invention encompasses the compounds in any racemic, optically-active, polymorphic, or stereroisomeric forms, or mixtures thereof. In one embodiment, the deuterated phospholipid compounds may include pure (R)-isomers. In another embodiment, the deuterated phospholipid compounds may include pure (S)-isomers. In another embodiment, the deuterated phospholipid compounds may include a mixture of the (R) and the (S) isomers. In another embodiment, the deuterated phospholipid compounds may include a racemic mixture comprising both (R) and (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The compounds of the invention can exist in unsolvated as well as solvated forms, including hydrated forms, e.g., hemi-hydrate. In general, the solvated forms, with pharmaceutically acceptable solvents such as water, ethanol, and the like are equivalent to the unsolvated forms for the purposes of the invention.

The term “deuterated phospholipid compound” also encompasses salt forms of the deuterated phospholipid compound.

Certain compounds of the invention also form pharmaceutically acceptable salts, e.g., acid addition salts. For example, the nitrogen atoms may form salts with acids. Examples of suitable acids for salt formation are hydrochloric, sulfuric, phosphoric, acetic, citric, oxalic, malonic, salicylic, malic, furmaric, succinic, ascorbic, maleic, methanesulfonic and other mineral carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66: 1-19 (1977).

Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

The compounds of the present invention can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Where the compounds according to the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention.

The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

“Subject” or “patient” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” or “patient” does not denote a particular age or sex.

As used herein, “administering” or “administration” includes any means for introducing cancer therapeutics, including radiotherapy and chemotherapy, into the body, preferably into the systemic circulation. Examples include but are not limited to oral, buccal, sublingual, pulmonary, transdermal, transmucosal, as well as subcutaneous, intraperitoneal, intravenous, and intramuscular injection.

A “diagnostically effective amount” means an amount of a compound that, when administered to a subject for screening for tumors, is sufficient to provide a detectable distinction between a benign structure and a malignant tumor. The “diagnostically effective amount” will vary depending on the compound, the condition to be detected, the severity or the condition, the age and relative health of the subject, the route and form of administration, the judgment of the attending medical or veterinary practitioner, and other factors.

A “therapeutically effective amount” means an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a patient for the purpose of combating the disease, condition, or disorder. Treating includes the administration of the compounds of present invention to alleviate the symptoms or complications, decrease the severity, or eliminate the disease, condition, or disorder.

The term “pharmaceutically acceptable” means that which is useful in preparing a pharmaceutical composition that is generally safe, non-toxic, and neither biologically nor otherwise undesirable and includes that which is acceptable for veterinary as well as human pharmaceutical use. The terms “pharmaceutically acceptable salts” or “prodrugs” includes the salts and prodrugs of compounds that are, within the scope of sound medical judgment, suitable for use with subjects without undue toxicity, irritation, allergic response, and the like, commensurate with a reasonable benefit/risk ratio, and effective for their intended use, as well as the zwitterionic forms, where possible, of the compounds.

As defined herein, “contacting” means that the deuterated phospholipid compound used in the present invention is introduced to a sample containing cells or tissue in a test tube, flask, tissue culture, chip, array, plate, microplate, capillary, or the like, and incubated at a temperature and time sufficient to permit binding of the deuterated phospholipid compound to a receptor or intercalation into a membrane. Methods for contacting the samples with the deuterated phospholipid compound or other specific binding components are known to those skilled in the art and may be selected depending on the type of assay protocol to be run. Incubation methods are also standard and are known to those skilled in the art.

In another embodiment, the term “contacting” means that the deuterated phospholipid compound used in the present invention is introduced into a patient receiving treatment, and the compound is allowed to come in contact in vivo. In further embodiment, the term “contacting” means that the deuterated phospholipid compound used in the present invention is introduced into a patient requiring screening for tumors, and the compound is allowed to come in contact in vivo.

The terms “phospholipid ether compound” and “phospholipid compound” are used interchangeably for the purposes of the present application.

The invention also generally relates to compositions comprising the compounds of the present invention.

As used herein, the term “composition” is intended to encompass a product comprising the specified ingredients in the specified amounts, as well as any product which results, directly or indirectly, from a combination of the specified ingredients in the specified amounts.

Compositions of the present invention may be prepared as a single unit dose or as a plurality of single unit doses. As used herein, a “unit dose” means a discrete amount of the composition comprising a predetermined amount of the active ingredient. The amount of the active ingredient is generally equal to the dosage of the active ingredient that would be administered to a patient or a fraction thereof.

As used herein, “pharmaceutical composition” means therapeutically effective amounts of the tumor-specific phospholipid ether analog together with suitable diluents, preservatives, solubilizers, emulsifiers, and adjuvants, collectively “pharmaceutically-acceptable carriers.” As used herein, the terms “effective amount” and “diagnostically effective amount” refer to the quantity of active agent sufficient to yield a desired effect without undue adverse side effects such as toxicity, irritation, or allergic response. The specific “effective amount” will vary with such factors as the particular condition being diagnosed, the physical condition of the subject, the species of the subject, the nature of concurrent therapy (if any), and the specific formulations employed and the structure of the compounds or its derivatives. The optimum effective amounts can be readily determined by one of ordinary skill in the art with routine experimentation.

Compositions of the present invention may be liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20™, Tween 80™, Pluronic F68™, bile acid salts), solubilizing agents (e.g., glycerol, polyethylene glycerol), anti-oxidants (e.g., ascorbic acid, sodium metabisulfite), preservatives (e.g., Thimerosal™, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

In a preferred embodiment, compositions of the present invention comprise a compound of the present invention, polysorbate, ethanol, and saline.

Also encompassed by the invention are methods of administering particulate compositions coated with polymers (e.g., poloxamers or poloxamines). Other embodiments of the compositions incorporate particulate forms protective coatings, protease inhibitors or permeation enhancers for various routes of administration, including topical, parenteral, pulmonary, nasal and oral. In some embodiments, the pharmaceutical composition is administered parenterally, paracancerally, transmucosally, tansdermally, intramuscularly, intravenously, intradermally, subcutaneously, intraperitonealy, intraventricularly, intracranially and intratumorally.

Further, as used herein “pharmaceutically acceptable carriers” are well known to those skilled in the art and include, but are not limited to, 0.01-0.1 M and preferably 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors. Other embodiments of the compositions according to the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparation can comprise the deuterated phospholipid compound alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the deuterated phospholipid compound can be administered to a patient by, for example, subcutaneous implantation of a pellet. In a further embodiment, a pellet provides for controlled release of tumor-specific phospholipid ether analog over a period of time. The preparation can also be administered by intravenous, intraarterial, or intramuscular injection of a liquid preparation oral administration of a liquid or solid preparation, or by topical application. Administration can also be accomplished by use of a rectal suppository or a urethral suppository.

The pharmaceutical preparations administrable by the invention can be prepared by known dissolving, mixing, granulating, or tablet-forming processes. For oral administration, the tumor-specific phospholipid ether analogs or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intra-arterial, or intramuscular injection), the tumor-specific phospholipid ether analogs or their physiologically tolerated derivatives such as salts, esters, N-oxides, and the like are converted into a solution, suspension, or expulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. Active therapeutic ingredients are often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

### Methods of Use

The compounds of the present invention may be used in a variety of diagnostic and therapeutic methods.

In one embodiment, the compounds may administered to the patient via either the enteral or parenteral routes (i.e., orally or via IV) for the therapeutical treatment of solid malignancies. The preferred malignancies include lung cancer, squamous cell carcinoma, renal cancer, adrenal cancer, melanoma, colon cancer, colorectal cancer, ovarian cancer, prostate cancer, liver cancer, intestinal cancer, hepatocellular carcinoma, retinoblastoma, cervical cancer, glioma, breast cancer, and pancreatic cancer.

In another embodiment, the compounds may administered to the patient via either the enteral or parenteral routes (i.e., orally or via IV) for the determination of the presence of internal malignancy. Examples include, but are not limited to, diagnosis of malignancy in the colon, rectum, small bowel, esophagus, stomach, duodenum, uterus, pancreas and common bile duct, bronchi, esophagus, mouth, sinus, lung, bladder, kidney, abdominal cavity or thoracic (chest) cavity.

The singular articles “a”, “an,” and “the” include plural reference unless specifically indicated or unless it is clear from the context that only the singular form is possible.

The invention will further be described with the following examples. These examples are described for illustrative purposes only and should not be deemed to narrow or limit the scope of the present invention.

### EXAMPLES

### Example 1

### Synthesis of CLR1401-d₉ (1)

For example, the synthesis of a deuterated version of CLR1401 can be performed as follows:

18-(p-Iodophenyl)-octadecanol (7) was converted into the cyclic phosphotriester (9) by reaction with 2-chloro-2-oxo-1,3,2-dioxaphospholane (8) in the presence of triethylamine. In the next step, the nucleophilic ring opening of the cyclic phosphotriester (9) with lithium bromide gave the 2-bromoethyl phosphodiester intermediate 10 (U. F. Heiser, B. Dobner, J. Chem. Soc., Perkin Trans. 1, 1997, 809-815). The quaternization of trimethylamine-d₉ with this intermediate provided CLR1401-d₉ (1) with purity of 99.4% (after chromatographic purification).

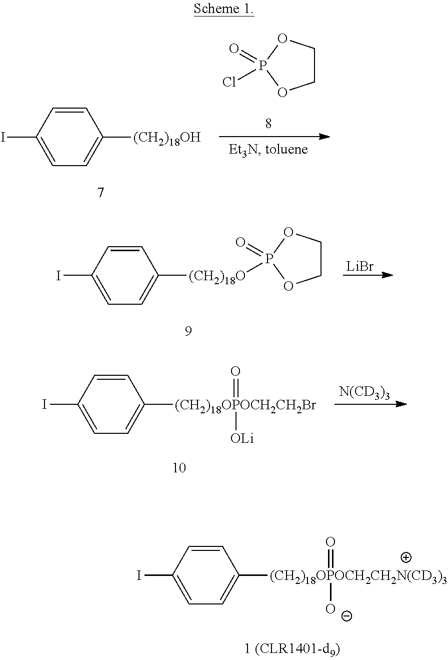

### Example 2

### Synthesis of CLR1401-d₁₃ (3)

### CLR1401-d (3)

While the other deuterated compounds have not yet been synthesized, it is envisioned that they can be easily synthesized with a reasonable expectation of success using known methods and the following teachings of the present invention. For example, synthesis of CLR1401-d₁₃ (3) with deuterated choline part of the molecule is shown below (Scheme 2). First, deuterated ethylene glycol (11) is converted into deuterated 2-chloro-2-oxo-1,3,2-dioxaphospholane-d₄ (13) according to the published procedure (T. Laube, H. Kurreck, J. Labelled Compd. Radiopharm., 1983, 20, 111-129). The rest of the synthesis is similar to the one for CLR1401-d₉.

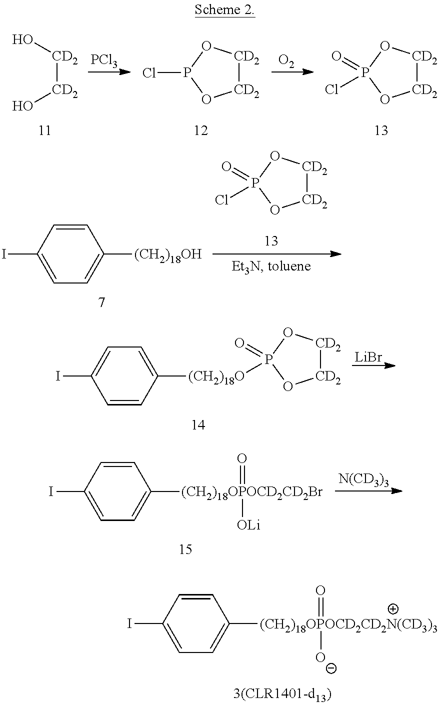

We claim:

1. A deuterated phospholipid compound selected from the group consisting of: 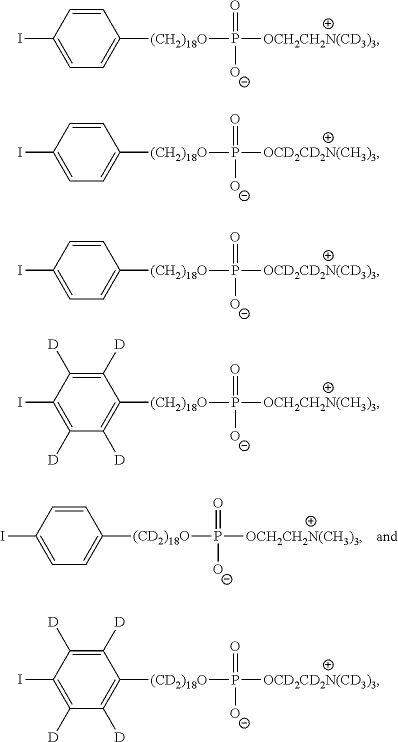
where I is a radioactive or stable isotope of iodine.

2. A composition comprising the deuterated phospholipid compound according to claim 1.

3. A method for the treatment of solid cancer in a subject having a solid cancer comprising administering to the subject a therapeutically effective amount of the deuterated phospholipid compound according to claim 1.

4. The method of claim 3 wherein said solid cancer is selected from the group consisting of lung cancer, squamous cell carcinoma, renal cancer, adrenal cancer, melanoma, colon cancer, colorectal cancer, ovarian cancer, prostate cancer, liver cancer, intestinal cancer, hepatocellular carcinoma, retinoblastoma, cervical cancer, glioma, breast cancer, and pancreatic cancer.

5. The method of claim 3, wherein said deuterated phospholipid compound has the following formula: 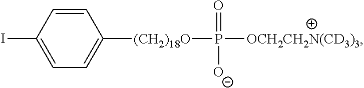
wherein I is ¹³¹I.

6. A method of detecting solid cancer in a subject suspected of having a solid cancer comprising administering to said subject a diagnostically effective amount of the deuterated phospholipid compound according to claim 1.

7. The method of claim 6, wherein said deuterated phospholipid compound has the following formula: 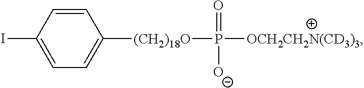
wherein I is ¹²⁴I.
